(19) United States
(12) Patent Application Publication
(10) Pub. No.: US 2016/0101177 A1
(43) Pub. Date: Apr. 14, 2016

(54) pH AND LIGHT ACTIVATED ANTI-CANCER DRUGS

(71) Applicant: The Board of Trustees of the University of Alabama, Tuscaloosa, AL (US)

(72) Inventors: Elizabeth T. Papish, Tuscaloosa, AL (US); Jared J. Paul, Villanova, PA (US); Edward J. Merino, Cincinnati, OH (US)

(21) Appl. No.: 14/882,675

(22) Filed: Oct. 14, 2015

Related U.S. Application Data
(60) Provisional application No. 62/063,613, filed on Oct. 14, 2014.

(51) Int. Cl. A61K 41/00; A61N 5/06; C07F 15/00

(52) U.S. Cl. CPC A61K 41/0042; C07F 15/0053; A61N 5/062

(57) ABSTRACT

Described are compositions of metal complexes that can be selectively activated by light when the metal complex is under acidic conditions, such as in a cancer cell. In some aspects, the metal complex can be utilized in a drug formulation with anti-cancer activity.

### CROSS-REFERENCE TO RELATED APPLICATION

This application claims priority to U.S. provisional application Ser. No. 62/063,613 filed on Oct. 14, 2014.

### STATEMENT REGARDING FEDERALLY SPONSORED RESEARCH

This invention was made with government support under Grant No. 1UL1RR026314-01 awarded by the National Institutes of Health and Grant No. CHE-0846383 awarded by the National Science Foundation. The government has certain rights in the invention.

### FIELD

The subject matter disclosed herein generally relates to anti-cancer drugs that can be selectively activated by pH and light. Also the subject matter disclosed herein generally relates to metal complexes, which can be utilized as anti-cancer drugs that can be selectively activated by pH and light.

### BACKGROUND

Metal-based anti-cancer agents can owe both their efficacy and their troublesome side effects to their inherent reactivity with DNA. Metal complexes can be effective anti-cancer drugs because they will bind to nucleic acids, which can result in halting further DNA transcription, replication, and ultimately cell death. Thus, a metal complex anti-cancer drug activity can be limited by the disassociation of one or more ligands, which frees up space for the metal to bind to the nucleic acid and ultimately cause cell death.

Many platinum-based metal complexes can be effective anti-cancer drugs based on their high reactivity with DNA, but this reactivity does not distinguish between cancer and non-cancer cells. For example, cisplatin can form cross-links with nucleic acids to halt DNA replication and transcription. However, a major problem with these agents are so called “off-target” effects wherein non-cancer cells can be effected by these DNA modification reactions. The indiscriminate reactivity of cisplatin can lead to drug induced toxicity, side effects, and little margin for error between the therapeutic dose and the toxic dose. More recently other strategies have attracted attention.

Ruthenium has been tested as a possible replacement for platinum in metal-based cancer drugs to lower the overall reactivity of the drug. Even though two ruthenium-based complexes have entered clinical trials (NAMI-A and KP1019), ruthenium based drugs can have a lower overall reactivity than traditional platinum-based anti-cancer drugs. This can be due to a slow ligand disassociation rate from the ruthenium metal center.

Some strategies have already been proposed to help increase the anti-cancer activity of metal-based drugs, such as photo-driven activation. Here, light can be used as a switch to turn on the reactivity of the drug once it has been incorporated into a cell. This has been previously demonstrated with platinum (Farrer et al. Chem. Res. Toxicology. 23 (2010) 413-421) and ruthenium (Howerton et al. J. Am. Chem. Soc. 134 (2012) 8324-8327).

One key process in the design of the new prodrug strategy is that Ru(II)-polypyridyl complexes are relatively inert but ligand dissociation can occur in the excited state (FIG. 1). In one study, Howerton et al. utilized a 6,6′-dimethyl-2,2′-bipyridine (66′bpy(Me)₂) ligand to enhance the photo-dissociation properties of the complex. The substitution at the 6,6′-position leads to a less strongly bound ligand that can exchange more readily upon excitation.

While the light-driven activation of a ruthenium complex can lead to anti-cancer activity, it can still suffer the same drawbacks as its platinum predecessors, such as indiscriminate cell reactivity. Thus, even if the ruthenium complex can be activated by light, it can still bind to any nearby nucleic acid molecule regardless if the nucleic acid molecule is within a cancer cell or not. One possible solution to this would be develop a tunable catalyst that can only be activated by light in a cancer cell. The compositions and methods disclosed herein address these and other needs.

### SUMMARY

In accordance with the purposes of the disclosed subject matter, as embodied and broadly described herein, this disclosure, in one aspect, relates to compositions and methods of making and using said compositions. In more specific aspects, the subject matter disclosed herein generally relates to anti-cancer drugs that can be selectively activated by pH and light. Also the subject matter disclosed herein generally relates to metal complexes, which can be utilized as anti-cancer drugs that can be selectively activated by pH and light. In more specific aspects, the disclosed metal complexes can be selectively activated by light when the complex is under acidic conditions, such as, but not limited to, in a cancer cell. In some aspects, the disclosed metal complexes can comprise an adjustable bipyridine ligand that can be designed to disassociate under a particular set of conditions. In some further aspects, the disclosed metal complexes can comprise additional ligands that can be chosen for particular advantages. In some further aspects, the disclosed metal complexes can be utilized in a drug formulation with anti-cancer activity. Methods of making and using metal complexes are also disclosed.

Additional advantages of the disclosed compositions and methods will be set forth in part in the description which follows, and in part will be obvious from the description. The advantages of the disclosed compositions will be realized and attained by means of the elements and combinations particularly pointed out in the appended claims. It is to be understood that both the foregoing general description and the following detailed description are exemplary and explanatory only and are not restrictive of the disclosed compositions, as claimed.

### BRIEF DESCRIPTION OF THE FIGURES

The accompanying figures, which are incorporated in and constitute a part of this specification, illustrate several aspects described below.

FIG. 1 displays the proposed mechanism for ligand dissociation upon absorption of light. R═CH₃ for Howerton et al. and R═OH for studies carried out in this work. * represents the excited state of the complex.

FIG. 2 displays the structures of the protonated and deprotonated forms of the ligands 4,4′-dihydroxy-2,2′-bipyridine (44′bpy(OH)₂) (a) and 6,6′-dihydroxy-2,2′-bipyridine (66′bpy(OH)₂) (b).

FIG. 3 demonstrates the light-induced HeLa cytotoxicity from ruthenium complexes. When HeLa cells were treated with [Ru(bpy)₂(66′bpy(OH)₂)]²⁺ or irradiated at 450 nm for 1 hour little cell death occurred. In contrast, when cells were treated with [Ru(bpy)₂(66′bpy(OH)₂)]²⁺ and 450 nm irradiation viability dropped to 47%. Controls using [Ru(bpy)₂(44′bpy(OH)₂)]²⁺ and [Ru(bpy)₃]²⁻ showed limited cell death, even with irradiation. (−) Indicates no irradiation and (+) indicates irradiation at 450 nm for 1 hour.

FIG. 4 displays the crystal structures of [Ru(bpy)₂(66′bpy(OH)₂)][PF₆]₂ (a) and [Ru(bpy)₂(66′bpy(O⁻)₂)](b). Counter ions are omitted for clarity.

FIG. 5 displays the cyclic voltammogram of (black line) 1.3 mM [Ru(bpy)₂(66′bpy(OH)₂)]²⁺ and (grey line) 1.1 mM [Ru(bpy)₂(66′bpy(O⁻)₂)] in acetonitrile with 0.1 M tetrabutylammonium hexafluorophosphate at 25° C. Tetrabutylammonium hydroxide was used for deprotonation. Scan rates were 200 mV/s. Data reported versus SCE standard.

FIG. 6 is a UV/Visible spectra of 75 μM [Ru(bpy)₂(66′bpy(OH)₂)]²⁺ in aqueous buffers ranging from pH=1 to pH=13 at 25° C.

FIG. 7 displays a graph of the highest occupied molecular orbitals and relative energies involved in electronic transitions for [Ru(bpy)₂(66′bpy(OH)₂)]²⁺ (a) and [Ru(bpy)₂(66′bpy(O⁻)₂)] (b).

FIG. 8 is a UV/Visible spectra of protonated 50 μM [Ru(bpy)₂(66′bpy(OH)₂)]²⁺ at pH 5 in aqueous media (a) and deprotonated 50 μM [Ru(bpy)₂(66′bpy(O⁻)₂)] at pH 7.5 in aqueous media (b) irradiated with 450 nm blue light from 0 minutes to 60 minutes. Protonated 50 μM [Ru(bpy)₂(66′bpy(OH)₂)]²⁺ in acetonitrile irradiated with 450 nm blue light from 0 minutes to 20 minutes is also shown (c). All spectra were collected at 25° C.

### DETAILED DESCRIPTION

The materials, compounds, compositions, articles, and methods described herein can be understood more readily by reference to the following detailed description of specific aspects of the disclosed subject matter and the Examples and Figures included herein.

Before the present materials, compounds, compositions, articles, devices, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an ionic liquid” includes mixtures of two or more such ionic liquids, reference to “the compound” includes mixtures of two or more such compounds, and the like.

“Optional” or “optionally” means that the subsequently described event or circumstance can or cannot occur, and that the description includes instances where the event or circumstance occurs and instances where it does not.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Unless specifically stated, a substituent that is said to be “substituted” is meant that the substituent is substituted with one or more of the following: alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol. In a specific example, groups that are said to be substituted are substituted with a protic group, which is a group that can be protonated or deprotonated, depending on the pH.

“A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkyl alcohol” is used in another, it is not meant to imply that the term “alkyl” does not also refer to specific terms such as “alkyl alcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is a group that contains an aromatic group that has at least one heteroatom incorporated within the ring of the aromatic group. Examples of heteroatoms include, but are not limited to, nitrogen, oxygen, sulfur, and phosphorus. Examples of heteroaryl groups include pyrrolyl, pyrrolinyl, imidazolyl, pyrazolyl, pyridyl, pyrimidinyl, pyrazinyl, pyridazinyl, imidazolyl, triazinyl, triazolyl, tetrazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heteroaryl groups include carbazolyl, benzidolyl, phenanthrolinyl, dibenzofuranyl, acridinyl, phenanthridinyl, xanthenyl and the like. The heteraryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol, as described below.

Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol as described herein. The term “biaryl” refers to two aryl groups that are bound together via a fused ring structure, as in naphthalene, or are attached via one or more carbon-carbon bonds, as in biphenyl or bipyridinyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻. The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above. The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above. The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN

The term “azido” as used herein is represented by the formula —N₃.

The term “silyl” as used herein is represented by the formula —SiA¹A²A³, where A¹, A², and A³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfo-oxo” is used herein to generally refer to sulfonyl, sulfonylamino, sulfonate, sulfonic acid, and sulfinyl groups. The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. The term “sulfonylamino” or “sulfonamide” as used herein is represented by the formula —S(O)₂NH—. The term “sulfonate” is used herein to refer to —SO₃—, which is a deprotonated sulfonic acid moiety —SO₃H. “Sufonate” can also refer to the sulfonate salt, e.g., the Li, Na, K, Ca, Mg, NH₄, salt even though the particular counterion may not be specified. The term “sulfinyl” as used herein refers to S(O)A¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

The term “thio” as used herein is represented by the formula —S—.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the subs

The term “complex” describes a coordination complex, which is a structure comprised of a central atom or molecule that is weakly connected to one or more surrounding atoms or molecules, or describes chelate complex, which is a coordination complex with more than one bond.

A “ligand” is an ion or molecule that binds to a central metal atom to form a coordination complex.

The term “prodrug” describes a drug molecule, which is a precursor drug molecule that is converted into the active form within the body through a chemical transformation, ligand disassociation, or proton transfer among others.

The term “nucleic acid” describes a polymeric macromolecule comprised of nucleotides. Some nucleic acids can be as deoxyribonucleic acid (DNA) or ribonucleic acid (RNA).

“Replication” is the process where a single DNA molecule is used to create two identical DNA molecules.

“Transcription” is the process where a DNA molecule is converted into an RNA molecule.

The term “pH” is a measure of the acidity or basicity of an aqueous solution. The pH is defined as the decimal logarithm of the reciprocal of the hydrogen ion activity in a solution.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Compositions

One way to distinguish cancer cells from non-cancer cells is pH. Cancer cells are more acidic (˜pH=5.0-6.5) than normal cells (˜pH=7.2-7.4) due to their high metabolism. A drug that can be selectively activated at low pH can selectively target cancer cells without many of the negative side effect of other non-selective drugs, such as drug induced toxicity, side effects, and little margin for error between the therapeutic dose and the toxic dose. The subject matter disclosed herein is a class of drugs that can only be activated while under the acidic conditions typical of a cancer cell. Disclosed herein is a selective ligand that can preferentially dissociate from a metal core when it is in a cancer cell.

Provided herein are metal complexes that comprise at least one metal atom and at least one ligand, wherein the ligand can disassociate from the metal atom upon contact with light in acidic conditions. Once the ligand has disassociated from the metal atom, the metal atom can bind to a nucleic acid molecule to prevent replication and transcription of the nucleic acid molecule. The cessation of nucleic acid replication results in cell death. Since cancer cells can be more acidic than non-cancer cells, the ligand can disassociate from the metal core in cancer cells, but potentially not in non-cancer cells. Thus, the metal complex can act as a selective anti-cancer compound.

In one aspect, disclosed herein are compositions of metal complexes. The use of the term “metal complex” is meant a compound that has at least one metal atom surrounded by one or more ligands. The metal complex, described herein, is stable when contacted with light under a neutral pH, or one that would be commonly contacted in a healthy cell (˜7.2-7.4), but would disassociate one or more of its ligands upon contact with light if dissolved in an acidic solution (˜pH=5.0-6.5 or lower). Reference herein to whether a ligand is protonated or deprotonated at a given pH refers to the ligand in the metal complex, not the free ligand.

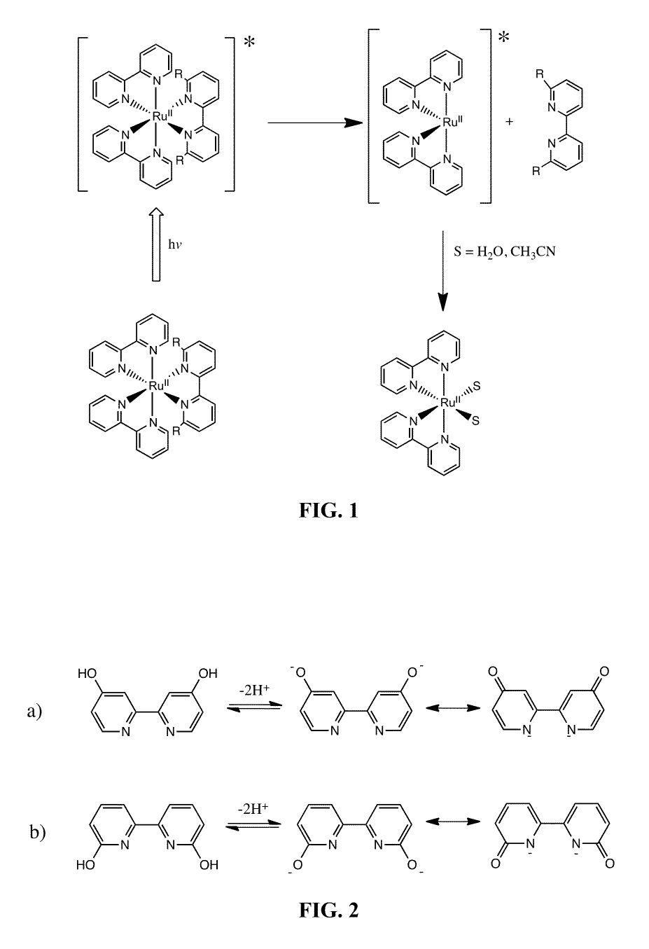

For example, as seen in FIGS. 1 and 2, the 66′bpy(OH)₂ ligand can be reversibly modified by changing the relative acidity of the solution. When the pH of the system is below 5.26, the hydroxyl groups attached to the bipyridine ligand in [Ru(bpy)₂(66′bpy(OH)₂)]²⁺ can be protonated, while above 7.27, the hydroxyl groups can be deprotonated. When the hydroxyl groups are protonated, the ligand can rapidly disassociate from the metal center. The metal center can then bind to DNA and stop cell reproduction. However, when the hydroxyl groups are deprotonated, the bipyridine ligand can be difficult to disassociate, which can lead to less metal complexes binding to DNA.

In one aspect, the metal atom in the metal complex can be a transitional metal. In another aspect the metal atom can comprise a ruthenium, platinum, gold, lanthanum, rhodium, silver, iron, zinc, silver, iridium, palladium, osmium, manganese, magnesium, copper, cobalt, scandium, titanium, vanadium, chromium, copper, nickel, yttrium, zirconium, molybdenum, technetium, cadmium, rhenium, or tungsten. In another aspect, the metal complex can comprise multiple metal atoms.

The metal complex can have one, two, three, four, five, six, seven, or eight different ligands attached to a single or multiple metal core. The metal center can be charged or neutral. The ligands can be selected for particular properties, such as solubility, nucleic acid intercalation, Bronsted or Lewis acidity, or Bronsted or Lewis basicity, among others.

Ligands can be selected for nucleic intercalation by designing them to have bulky aromatic groups, which will separate strands of nucleic acid from one another. One example of such groups are linked aromatic groups. Some suitable ligands include but are not limited arenes with pi bonds, such as biphenyl, phenanthroline, anthracene, naphthalene, and pyrene.

The ligands can be bound to the metal center in many ways known to a person skilled in the art. Some examples of potential metal-ligand interactions include, but are not limited to an interaction of a metal atom or ion with a heteroatom, such as nitrogen, phosphorous, oxygen, chlorine, bromine, fluorine, sulfur, iodine, or silicon atom, a hydride molecule, directly to a carbon atom, or a π system.

A single ligand can interact with a metal atom or ion one (monodentate), two (bidentate), or three (tridentate) times.

Some classes of potential ligands include, but are not limited to amines, heteroaryl, aryl, phosphines, biaryls, halides, alkyl, alkenyl, and alkynyl groups among others. In another aspect the ligand can be specifically a substituted or substituted bipyridine molecule. In a specific example, the ligand can be a bipyridine substituted with one or more protic groups. In further examples, the ligand can be a bipyridine with the following structure:

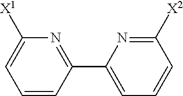

where X¹ and X² are protic groups, independently selected from —OH, —SH, —NH₂, —SO₃H, —CO₂H, and —SO₂NH₂.

Some other examples of suitable ligands are derivatives of bipyridine (bpy), dipyrido[3,2-f:2′,3′-h]-quinoxaline) (dpq), dipyrido[3,2-a:2′,3′-c]phenazine (dppz), or 3,6-bis(2′-pyridyl)pyridazine (dppn).

The metal complex can be optionally charged. In some aspects, the metal complex is positively charged. In other aspects, the metal complex is negatively charged. Thus, the metal complex can further comprise a counter ion. Some suitable examples of potential counter ions include, but are not limited to chloride, bromide, iodide, hexaflurophoshate, tetrafluroborate, carbonate, hydroxide, lithium, and sodium.

In certain examples, disclosed herein are metal complexes that have Formula I:

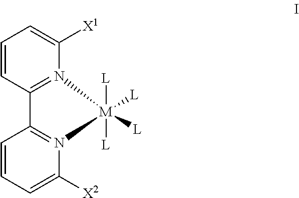

where M is a metal, X¹ and X² are protic groups, and each L is either a monodentate ligand or together with another L a bidentate ligand, as described herein. In certain examples, M is ruthenium. In certain examples, M is platinum. In other examples, X¹ and X² are independently, —OH, —SH, —NH₂, —SO₃H, —CO₂H, —SO₂NH₂, which depending on the pH can be deprotonated. In specific examples, X¹ and X² are OH. In other specific examples, two Ls are substituted or unsubstituted bipyridinyl, two L's are unsubstituted or unsubstituted phenanthroline, where when substituted they can be substituted with alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol. In other examples one or more L groups are substituted with a protic group, such as OH, —SH, —NH₂, —SO₃H, —CO₂H, —SO₂NH₂.

### Synthesis and Characterization

[Ru(bpy)₂(66′bpy(OH)₂)]²⁺ can be synthesized by treating [Ru(bpy)₂(Cl)₂] with the 66′bpy(OH)₂ ligand as described in the Examples. A similar protocol can be followed with other metal chloride complexes. The anti-cancer activity of the complexes can be determined as described herein. In some embodiments, the anti-cancer activity of the metal complex can be determined by examining if the complex is cytotoxic to HeLa cells (relative to the other Ru complexes). Several cancer cell lines can exhibit lower pH values due to their excessive metabolic activity, including HeLa cells. HeLa cells can have a pH of 6.5 in the golgi, when metabolizing glucose. HeLa can be a model to demonstrate the light-driven photo-dissociation of a ruthenium bound bipyridine ligand. In addition, Ru²⁺ and Pt²⁺ complexes can be cytotoxic when a ligand is lost to form the di-water complex. This di-water complex can exchange the water ligands with biomolecules to induce toxicity. Ligand exchange reactions can lead to the addition of the nucleobase guanine at the N7-positon. Nucleobase binding to metallodrugs can occur with both DNA and RNA. Ru²⁺-polypyridyl complexes, with slower water exchange rates, can have a means of actively dissociating the ligand. The design of prodrugs that can be activated selectively are useful in treating diseases such as cancer. The ability to discriminate between healthy cells and cancer cells could limit some of the adverse effects patients normally suffer from when being treated. Selective targeting of cancer cells can be achieved with this prodrug approach because cancer cells are typically more acidic than normal cells. The photo-dissociating ligand can be bound more weakly to the metal center when protonated, and this can allow the active cell-killing agent to be made in greater quantities in cancerous cells.

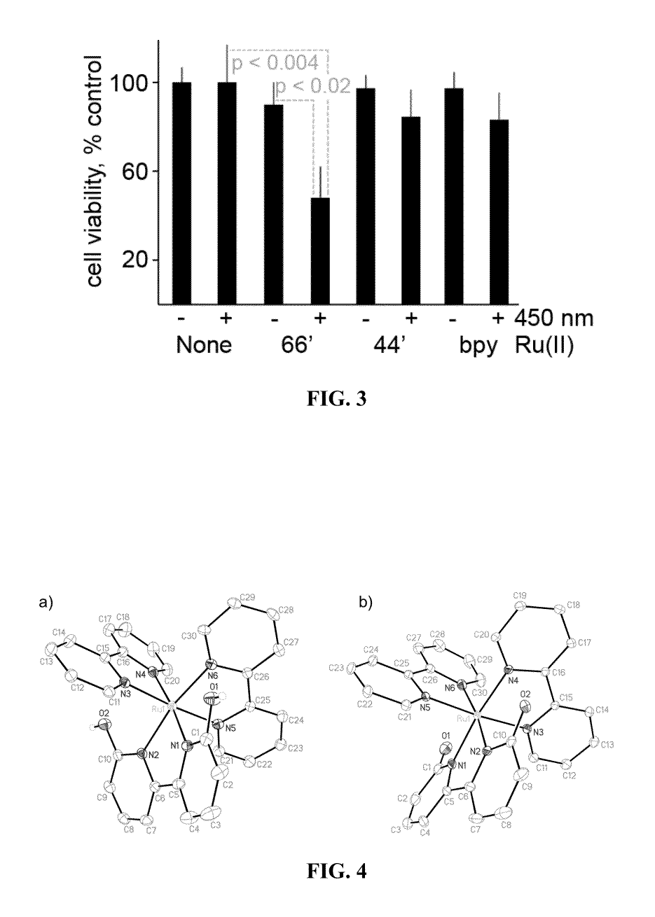

The anti-cancer activity of [Ru(bpy)₂(66′bpy(OH)₂)]²⁺, [Ru(bpy)₂(44′bpy(OH)₂)]²⁺ and [Ru(bpy)₃]²⁺ in HeLa cells is demonstrated in FIG. 3. Upon irradiation the [Ru(bpy)₂(66′bpy(OH)₂)]²⁺ prodrug can be converted into a cytotoxic agent while in a cell. In some embodiments, when the metal complex is incubated with cancer cells and irradiated, the viability of the cells can drop to 60% or less, 55% or less, 50% or less, or 45% or less.

### Methods of Use

Further provided herein are methods of treating or preventing cancer in a subject, comprising administering to the subject an effective amount of a compound or composition as disclosed herein. The methods can further comprise administering a second compound or composition, such as, for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent or an anti-inflammatory agent) or administering an effective amount of ionizing radiation to the subject.

Also provided herein are methods of photodynamic therapy of tumors, comprising contacting the tumor with an effective amount of a compound or composition as disclosed herein and irradiating the tumor with an effective amount of light.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. Oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (liver) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenström's macroglobulinemia, and Wilms' tumor.

In some aspect, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during or after surgical intervention to remove all or part of a tumor. Administration may be accomplished via direct immersion; systemic or localized intravenous (i.v.), intraperitoneal (i.p.), subcutaneous (s.c.), intramuscular (i.m.), or direct injection into a tumor mass; and/or by oral administration of the appropriate formulations.

A cancer immunotherapeutic agent suitable for use in the methods disclosed herein is an immunotherapeutic agent which comprises a cell effector component joined to a tumor associated antigen targeting component. Suitable cell effector components can include cytotoxic chemicals, cytotoxic radioisotopes, and cell signaling agents such as cytokines. Suitable tumor targeting components are polypeptide chains which bind to tumor associated antigens present on or in the surrounding tissue matrix of a tumor cell such as receptor protein chains or immunoglobulin chains.

Tumor associated antigens which can be used for targets of the immunotherapeutic agents include a tumor associated antigen selected from the group consisting of AFP, CA 125, CEA, CD19, CD20, CD44, CD45, EGF Receptor, GD[2], GD[3], GM1, GM2, Her-2/Neu, Ep-CAM (KSA), IL-2 receptor, Lewis-Y, Lewis-X (CD 15), melanoma-associated proteoglycan MCSP, PSA and Transferrin Receptor.

Examples of immunotherapeutic agents have an effector component that is a cytokine polypeptide joined to a targeting component which is an immunoglobulin (Ig) polypeptide chain. The Ig polypeptide chain comprises a variable region which binds to a tumor associated antigen. It is preferred that said immunoglobulin chain, when combined with the appropriate complementary chain (i.e. a heavy chain complements a light chain) defines an antibody active site which is specific for a tumor associated antigen.

The tumor targeting Ig portion of the immunotherapeutic agent can comprise an entire immunoglobulin chain amino acid sequence, or at least the fragment of which comprises the antigen binding specificity portion of the protein. Thus, a suitable Ig polypeptide chain will have at least an Ig variable region specific for a tumor associated antigen.

An antibody and polypeptide chains therefrom, suitable for use in the disclosed methods, will have an amino acid sequence that can be of any mammalian origin. Where such antibody protein is not of the same origin as the anticipated patient, fragments of the antibody protein, such as F(ab′)2, Fab, Fv or engineered Fv single chain antibody protein can be used. To further reduce antigenicity of the antibody protein, modification of the antibody amino acid sequence may be accomplished to reduce such by making the protein appear more like the patients normal antibody components. For example, monoclonal murine antibody amino acid sequences can be modified to appear more human, for administration to human patients by a variety of processes for humanization of the antibody.

Specific examples of cancer immunotherapeutic agents include an antibody that specifically binds CLTA-4, such as ipilimumab (Bristol-Myers Squibb), anti-PD-1, anti-PDL1. Other immunotherapeutic agents include the TNFα antagonists (e.g. etanercept), the B cell depleting agent rituximab, the anti-IL-6 receptor tocilizumab, and the costimulation blocker abatacept can be administered with the compounds or compositions disclosed herein.

The disclosed compounds can also be administered with toll like receptor (TLR) agonist. TLR agonist is a ligand for a TLR selected from the group consisting of TLR1, TLR2, TLR3, TLR4, and TLR9. For example, the TLR agonist can be a ligand selected from the group consisting of Pam3CSK4, Pam3CSK4, poly I:C, Ribomunyl, and CpG ODN.

The disclosed compounds can also be administered with an angiogenesis inhibiting agent, which is one which can inhibit the formation of new blood vessels (neovascularization) or enlargement of existing capillary networks into the tissues near a tumor cell. Suitable angiogenesis inhibiting agents can be peptides with angiogenesis inhibiting activity, such as the tumor associated antigen PSA. Other suitable angiogenesis inhibiting agents can be antagonists of VEGF associated angiogenesis, for example antagonists of the VEGF receptor on the surface of cells. One monoclonal antibody which can be used is LM609 (ATCC HB 9537).

Administration

The disclosed compounds can be administered either sequentially or simultaneously in separate or combined pharmaceutical formulations. When one or more of the disclosed compounds is used in combination with a second therapeutic agent, the dose of each compound can be either the same as or differ from that when the compound is used alone. Appropriate doses will be readily appreciated by those skilled in the art.

The term “administration” and variants thereof (e.g., “administering” a compound) in reference to a compound as described herein means introducing the compound or a prodrug of the compound into the system of the animal in need of treatment. When a compound as described herein or prodrug thereof is provided in combination with one or more other active agents (e.g., a cytotoxic agent, etc.), “administration” and its variants are each understood to include concurrent and sequential introduction of the compound or prodrug thereof and other agents.

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. No. 4,608,392; U.S. Pat. No. 4,992,478; U.S. Pat. No. 4,559,157; and U.S. Pat. No. 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation; East Hanover, N.J.) and HERCEPTIN (Genentech, Inc.; South San Francisco, Calif.), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan. Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., I¹³¹, I¹²⁵, Y⁹⁰, P³², etc.), and toxins of bacterial, fungal, plant, or animal origin (e.g., ricin, botulinum toxin, anthrax toxin, aflatoxin, jellyfish venoms (e.g., box jellyfish, etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

Reagents were obtained from Aldrich Chemical Company and used without further purification. RuCl₃.3H₂O was purchased from Pressure Chemical Company. [Ru(bpy)₂(Cl)₂], [Ru(bpy)₃][Cl]₂, [Ru(bpy)₂(CH₃CN)₂][PF₆]₂, and [Ru(bpy)₂(44′bpy(OH)₂)][PF₆]₂ were synthesized according to previously published methods. (Klein et al. Inorg. Chem. 50 (2011) 2754-2763). The 66′bpy(OH)₂ ligand was synthesized according to previously published methods. (Nieto et al. Organometallics. 30 (2011) 6339-6342.) For studies carried out in water, ruthenium hexafluorophosphate salts were converted to chloride salts by precipitation from acetone using tetrabutylammonium chloride dissolved in acetone. Aqueous solutions were prepared using a Millipore DirectQ UV water purification system.

¹H-NMR spectra were collected on a Varian 300 MHz Fourier Transform spectrometer in deuterated acetonitrile (CD₃CN) Infrared spectra were obtained on a Perkin Elmer Spectrum One FT-IR with Universal ATR sampling accessory. UV-visible absorption spectra were collected on a Scinco S-3100 diode-array spectrophotometer at a resolution of 1 nm. Luminescence data was collected on a Horiba Jobin Yvon Fluoromax 3. pH measurements were performed using a VWR SympHony pH meter, utilizing a three point calibration at pH=4.0, 7.0, and 10.0.

Electrochemical measurements were carried out on a Bioanalytical Systems (BAS) CW-50 potentiostat. A standard three electrode setup with a Ag/Ag⁺ reference electrode, platinum wire auxiliary electrode and glassy carbon working electrode were used. All measurements were taken in 0.1 M tetrabutylammonium hexafluorophosphate (TBAPF₆) in acetonitrile electrolyte solution. The solutions were degassed for approximately 20 minutes with argon before data collection. Ferrocene was used as an internal standard with E_{1/2}=+0.40 V vs. Saturated Calomel Electrode (SCE).

### Example 1

### [Ru(bpy)₂(66′bpy(OH)₂)][PF₆]₂.H₂O

A round bottom flask containing 30 mL of 1:1 ethanol:water was degassed with argon for 30 minutes. To the flask, 0.2260 g (1.201 mmol) 66′bpy(OH)₂ and 0.4843 g (0.9999 mmol) Ru(bpy)₂(Cl)₂ were added. The reaction mixture was heated at 80° C. under argon for 12 h. The reaction mixture turned red in color. After heating, the reaction mixture was allowed to cool to room temperature and filtered to remove any insoluble, unreacted ligand. A few drops of concentrated HCl was added to the filtrate to ensure protonation and the solution was diluted to 200 mL with water. An aqueous solution of ammonium hexafluorophosphate was added to the filtrate to precipitate the complex as the hexafluorophosphate salt. The complex was filtered and rinsed with copious amounts of water and allowed to air dry overnight. Yield: 0.5738 g (0.6309 mmol), 63%. δH (300 MHz, CD₃CN): δ 6 8.70 (broad), δ 8.50 (d, 2H), δ 8.35 (d, 2H), δ 7.95 (m, 10H), δ 7.55 (d, 2H), δ 7.45 (t, 2H), δ 7.20 (t, 2H), δ 6.70 (d, 2H). Elem. Anal: Found: C, 39.43; N, 9.23; H, 2.89%. Calc. for RuC₃₀N₆O₂H₂₄P₂F₁₂.H₂O: C, 39.62; N, 9.24; H, 2.88%.

### Example 2

### [Ru(bpy)₂(66′bpy(OH)₂)][PF₆]₂

Crystals of [Ru(bpy)₂(66′bpy(OH)₂)][PF₆]₂ were grown by the slow diffusion of ether into a benzonitrile solution with dissolved complex. The solutions were shielded from the light using aluminum foil. A single red block (0.08×0.12×0.13 mm) was mounted using NVH immersion oil (Cargille Laboratories) onto a nylon fiber and cooled to the data collection temperature of 110(2) K. Data were collected on a Brüker-AXS Kappa APEX II CCD diffractometer with 0.71073 Å Mo-Ka radiation. Unit cell parameters were obtained from 60 data frames, 0.5° Φ, from three different sections of the Ewald sphere yielding a=15.661(2), b=16.821(2), c=19.071(2) Å, α=107.42(1), β=100.60(1), γ=105.36(1)°, V=4429(1) Å³. 94387 reflections (Rᵢₙₜ=0.0534) were collected (33738 unique) over θ=1.41 to 33.19°. The data was consistent with the centrosymmetric, triclinic space group P-1. The data-set was treated with SADABS absorption corrections based on redundant multi-scan data, Tₘₐₓ/Tₘᵢₙ=1.03. The asymmetric unit contains two [Ru(bpy)₂(66′bpy(OH)₂)]²⁺ cations, four [PF₆]⁻ anions, four molecules of diethylether solvent and ½ a molecule of benzonitrile solvent. Two of the ether molecules are disordered over two positions, which were located from the difference map and refined using SIMU, DELU, and SAME commands. The benzonitrile molecule is disordered about the inversion center. One of the two positions was located from the difference map and the occupancy of all atoms was set to 0.5 along. A PART-1 command was used to ignore the symmetry at this position. The O—H protons were originally located from the difference map but three of them would not survive a free refinement. They were refined using a riding model with O—H bond distance refinement for possible H-bonding interactions to be considered. There is some minor residual density remaining around one of the [PF₆]⁻ anions which is due to positional disorder. All non-hydrogen atoms were refined with anisotropic displacement parameters. All other hydrogen atoms were treated as idealized contributions. The goodness of fit on F² was 1.019 with R1(wR2) 0.0551(0.1231) for [Iq>2(I)] and with largest difference peak and hole of 1.694 and −1.147 e/Å³ due to heavy atom noise around the ruthenium atom.

### Example 3

### [Ru(bpy)₂(66′bpy(O⁻)₂)]

Crystals of [Ru(bpy)₂(66′bpy(O⁻)₂)] were grown by the slow diffusion of ether into a solution containing acetonitrile with dissolved complex and a few drops of aqueous tetrabutylammonium hydroxide to ensure deprotonation of the complex. A single red block (0.10×0.18×0.18 mm) was mounted using NVH immersion oil onto a nylon fiber and cooled to the data collection temperature of 120(2) K. Data were collected on a Brüker-AXS Kappa APEX II CCD diffractometer with 0.71073 Å Mo-Ka radiation. Unit cell parameters were obtained from 60 data frames, 0.5° Φ, from three different sections of the Ewald sphere yielding a=9.411(1), b=12.287(1), c=13.003(1) Å, α=84.34(1), β→88.75(1), γ=73.88(1)°, V=1436.6(2) Å³. 26434 reflections (Rᵢₙₜ=0.0293) were collected (9615 unique) over θ=1.57 to 31.79°. The data was consistent with the centrosymmetric, triclinic space group P-1. The data-set was treated with SADABS absorption corrections based on redundant multi-scan data, Tₘₐₓ/Tₘᵢₙ=1.05. The asymmetric unit contains one [Ru(bpy)₂(66′bpy(O⁻)₂)] molecule, one molecule of acetonitrile solvent and one molecule of diethyl ether solvent. These solvent molecules were located from the difference map and were disordered over inversion centers. Attempts to model using PART-1 commands along with SIMU and DELU restraints resulted in unstable refinements so SQUEEZE was employed removing the electron density of the disordered solvent from the model. The molecular formula was augmented to include these solvent molecules. All non-hydrogen atoms were refined with anisotropic displacement parameters. All hydrogen atoms were treated as idealized contributions. The goodness of fit on F² was 1.024 with R1(wR2) 0.0368(0.0854) for [Iq>2(I)] and with largest difference peak and hole of 0.778 and −0.513 e/Å³.

### Computational Studies

All calculations were performed using GAMESS. Geometries were optimized using restricted B3LYP with the 6-31G* basis set for the main group elements. A scalar relativistic model core potential (first 30 electrons) was used for ruthenium, with the valence orbital set (5 s and 4d) being of triple-zeta quality. Spherical harmonic d orbitals were used in all calculations and the default grid size was used for numerical integration in DFT. The maximum tolerance for any nuclear gradient component was set to 0.0005 hartrees/bohr, and the default RMS gradient maximum was used (0.00017 hartrees/bohr). The nature of each stationary point was determined by running frequency calculations at the Density Functional Theory (DFT) level. Numerical frequencies were calculated via central differences of analytically determined energy gradients. For both structures all vibrational modes were found to be real at the DFT determined stationary points. Vertical excitation energies were calculated using time-dependent DFT (TDDFT) with the same set of functionals and basis sets used to characterize the ground state structures. Solvent effects on the vertical excitation energies were evaluated using the PCM solvation model. The solvated energies were evaluated at gas-phase optimized geometries. The solute cavity was determined using the simplified united atomic radii. The solvent considered in these calculations was water.

### Anti-Cancer Activity

For cell culture, human cervical carcinoma (HeLa) cells were obtained from the ATCC. The cells were cultured at 37° C. in a 5% CO₂, humidified atmosphere in Dulbecco's Modified Eagle Medium (DMEM) supplemented with 10% fetal bovine serum, 100 U/mL penicillin, and 100 μg/ml streptomycin (Invitrogen). Assays were accomplished by seeding cells at a density of 5,000 cells/well in a 96-well plate and incubated at 37° C. overnight. Media was exchanged and then cells were treated with either 100 μM [Ru(bpy)₂(66′bpy(OH)₂)]²⁺, [Ru(bpy)₂(44′bpy(OH)₂)]²⁺, or [Ru(bpy)₃]²⁺ for 48 h. The medium containing compounds was discarded, cells were washed, and fresh Hank's Balanced Salt Solution was added. In the dark, cells were irradiated using a 450 nm light emitting diode (LED) flashlight positioned 4 cm from the plate for 1 h. The salt solution was exchanged with media and cells were allowed to grow for 24 h. Media containing 20 μL of 3-(4,5-dimethylthiazol-2-yl)-2,2-diphenyltetrazolium bromide (MTT) (5 mg/mL) was added to each well and incubated for an additional 3 h. The medium was removed. After adding 200 μL of DMSO to each well, the optical densities at 570 nm were determined The p-values were calculated from triplicate data sets using KaleidaGraph software.

TABLE 1
IC₅₀ Values for Ruthenium Complexes.

| Complex | IC₅₀ (error), μM |
|---|---|
| [Ru(bpy)₂(66′bpy(OH)₂)]²⁺ | 88 (9) |
| [Ru(bpy)₂(44′bpy(OH)₂)]²⁺ | >100 |
| [Ru(bpy)₃]²⁺ | 152 (18) |

### Synthesis and Characterization

[Ru(bpy)₂(66′bpy(OH)₂)]²⁺ was synthesized by treating [Ru(bpy)₂(Cl)₂] with the 66′bpy(OH)₂ ligand. Anti-cancer activity of the complex was compared with two other complexes, [Ru(bpy)₂(44′bpy(OH)₂)]²⁺ and [Ru(bpy)_{3]}²⁺, which served as controls.

### Blue Light Driven Anti-Cancer Activity

The anti-cancer activity of [Ru(bpy)₂(66′bpy(OH)₂)]²⁺ was compared to [Ru(bpy)₂(44′bpy(OH)₂)]²⁺ and [Ru(bpy)₃]²⁺ by examining if [Ru(bpy)₂(66′bpy(OH)₂)]²⁺ could be cytotoxic to HeLa cells (relative to the other Ru complexes) since the hydroxyl-substituted bipyridyl ligand can disassociate from the metal complex at a low pH. Several cancer cell lines can exhibit lower pH values due to their excessive metabolic activity, including HeLa cells. HeLa cells can have a pH of 6.5 in the golgi, when metabolizing glucose. HeLa can be a model to demonstrate the light-driven photo-dissociation of a ruthenium bound bipyridine ligand. In addition, most Ru²⁺ and Pt²⁺ complexes can be cytotoxic when a ligand is lost to form the di-water complex. This di-water complex can exchange the water ligands with biomolecules to induce toxicity. Ligand exchange reactions can lead to the addition of the nucleobase guanine at the N7-positon. Nucleobase binding to metallodrugs can occur with both DNA and RNA. Ru²⁺-polypyridyl complexes, with slower water exchange rates, can have a means of actively dissociating the ligand. Thus, 66′bpy(OH)₂ was examined to see if it could be a trigger to allow for more rapid initiation of the exchange reactions upon light excitation.

The anti-cancer activity of [Ru(bpy)₂(66′bpy(OH)₂)]²⁺, [Ru(bpy)₂(44′bpy(OH)₂)]²⁺ and [Ru(bpy)₃]²⁺ in HeLa cells is demonstrated in FIG. 3. Upon irradiation the [Ru(bpy)₂(66′bpy(OH)₂)]²⁺ prodrug can be converted into a cytotoxic agent while in a cell. For example, HeLa cells were plated, incubated with [Ru(bpy)₂(66′bpy(OH)₂)]²⁺, irradiated, and an MTT cell viability assay was used to assess cellular viability. Untreated cells that do not receive any irradiation were taken as 100% viability. When HeLa are treated with [Ru(bpy)₂(66′bpy(OH)₂)]²⁺ in the absence of light, little cell death occurred. The average viability dropped to 93+/−6%. The change is not statistically significant with p greater than 0.05. Similar results were obtained when cells were not treated with the agent and only irradiated for one hour. The viability dropped to 88+/−9%. This shows that the ruthenium complex by itself is not cytotoxic. When [Ru(bpy)₂(66′bpy(OH)₂)]²⁺ was incubated with HeLa cells and irradiated the viability dropped to 47+/−12%. This is a statistically significant drop with p less than 0.004 and 0.02 for no ruthenium-complex and no irradiation respectively. Further experiments changing the concentration of [Ru(bpy)₂(66′bpy(OH)₂)]²⁺ in HeLa cells under irradiation can allow for measurement of an inhibitory concentration (IC₅₀). The IC₅₀ for anti-cancer activity is given in Table 1, which are consistent with the data in FIG. 3.

The relevance of the hydroxyls at the 6 and 6′ position of the bipyridine ligand was investigated by both re-orienting the hydroxyl groups to the 4 and 4′ position of the bipyridine and removing them completely. When the hydroxyl groups are removed the complex, [Ru(bpy)3]²⁺ is formed. When [Ru(bpy)₃]²⁺ was incubated with HeLa cells the viability was 91+/−4%. The [Ru(bpy)₃]²⁻ complex once incubated with HeLa cells, followed by irradiation results in a viability drop to 84+/−5%. When the hydroxyl groups were moved to the 4 and 4′ position, the viability was 91+/−4% without irradiation and 86+/−6% with irradiation. [Ru(bpy)₂(66′bpy(OH)₂)]²⁺ possessed high anti-cancer cell activity upon irradiation. Importantly, when the anti-proliferative effects of both [Ru(bpy)₂(44′bpy(OH)₂)]²⁺ and [Ru(bpy)₃]²⁺ on HeLa cells were investigated no significant effects were found for [Ru(bpy)₂(44′bpy(OH)₂)]²⁺ while [Ru(bpy)₃]²⁺ had an IC₅₀ value of 152 μM. Furthermore, the work described herein shows that the 66′bpy(OH)₂ ligand allows for both light triggered ligand release and pH sensitivity that can allow for selective toxicity towards more acidic (or cancerous) cells.

### X-Ray Structural Analysis Shows That Light Can Induce Ligand Displacement

The structures of [Ru(bpy)₂(66′bpy(OH)₂)][PF₆]₂ and the deprotonated form of the complex, [Ru(bpy)₂(66′bpy(O⁻)₂)] were determined by X-ray diffraction. The structure helps explain the propensity for 66′bpy(OH)₂ ligand dissociation. The two structures are depicted in FIG. 4. Early attempts to grow crystals suitable for X-Ray analysis were carried out by dissolving [Ru(bpy)₂(66′bpy(OH)₂)][PF₆]₂ in acetonitrile with slow diffusion of ether without protection from light. Crystal formation took several weeks and upon analysis, yielded the substituted complex, [Ru(bpy)₂(CH₃CN)₂][PF₆]₂ whereby two solvent molecules displaced the bidentate 66′bpy(OH)₂ ligand. The [Ru(bpy)₂(CH₃CN)₂][PF₆]₂ complex structure has been previously reported in the literature. (Heeg et al. Acta Crystallogr., Sect. C. 41 (1985) 684-686). Other crystals were also obtained from the crystallization that were clearly of a different type, but not resolvable. A lack of protection from light can lead to photo-substitution of ligand with solvent, which yielded the corresponding crystals with solvent replacing the 66′bpy(OH)₂. This result indicates that the [Ru(bpy)₂(66′bpy(OH)₂)]²⁻ complex does readily lose the 66′bpy(OH)₂ ligand upon irradiation and thus could be used as an anti-cancer prodrug.

To determine if light can cause ligand dissociation, crystals were grown by keeping the solutions in the dark and resulted in the desired complex, [Ru(bpy)₂(66′bpy(OH)₂)]²⁺ with two PF₆_{^{−}} counter ions. The complex takes on a distorted octahedral geometry with adjacent N—Ru—N bond angles ranging from 77.37(9)° to 98.95(9)°. This geometry is in accordance with other ruthenium hydroxyl-substituted-bipyridine complexes reported previously as well as the parent complex, [Ru(bpy)₃]²⁺. The Ru—N bond lengths associated with the unsubstituted bipyridine ligands range from 2.043(2) Å to 2.066(2) Å, which are also close in length to those bonds previously reported. However, the Ru—N bond lengths are elongated on average by ˜0.04 Å to 2.091(2) Å and 2.094(2) Å when the ligand is 66′bpy(OH)₂. This length is ˜0.3 Å longer than the Ru—N bond lengths for the 44′bpy(OH)₂ ligand substituted complex, [Ru(bpy)₂(44′bpy(OH)₂)][PF₆]₂. This longer bond length can be due to the orientation of the hydroxyl groups closer to the ruthenium center of the complex causing steric clashes that do not exist when the hydroxyl groups are oriented away from the metal center as with the 44′bpy(OH)₂ ligand. The longer and weaker bond between the Ru center and the 66′bpy(OH)₂ ligand can explain why this ligand is the one substituted in the complex upon light absorption.

Adding a few drops of aqueous tetrabutylammonium hydroxide to the crystallization solution yielded crystals of the corresponding deprotonated complex (structure (b) in FIG. 4). The overall structure of the complex does not change significantly upon deprotonation. The Ru—N bond lengths to both the unsubstituted bpy ligand and the 66′bpy(O⁻)₂ ligand do not vary significantly on average from the protonated form. The most noticeable bond length distance changes occurs as the C—O bonds decrease from 1.340(3) Å and 1.329(3) Å to 1.249(3) Å and 1.251(2) Å upon deprotonation. This decrease in bond length can be explained by the resonance structure wherein the C—O bonds take on double bond character upon deprotonation, structure (a) in FIG. 2. This decrease in bond length of ˜0.08 Å is slightly larger than the ˜0.05 Å bond length decrease observed in deprotonating [Ru(bpy)₂(44′bpy(OH)₂)]²⁺. However, in the deprotonated [Ru(bpy)₂(44′bpy(O⁻)₂)] complex there are several hydrogen bonded water molecules that most likely result in an elongation of the C—O bond, that is absent in the crystal structure of [Ru(bpy)₂(66′bpy(O⁻)₂)] where no hydrogen bonded solvent molecules are present. Although the Ru-N bond lengths remain longer for the Ru to 66′bpy(O⁻)₂ ligand, the complex does not appear to undergo as rapid photo-dissociation when exposed to 450 nm blue light compared to the protonated form. This result can be due to the significantly enhanced electron-donating effects of the ligand to the metal upon deprotonation. These effects were examined by computational means to help explain the lack of photo-dissociation of the deprotonated form upon irraditation.

### Cyclic Voltammetry Studies to Determine Ligand Effects on Metal

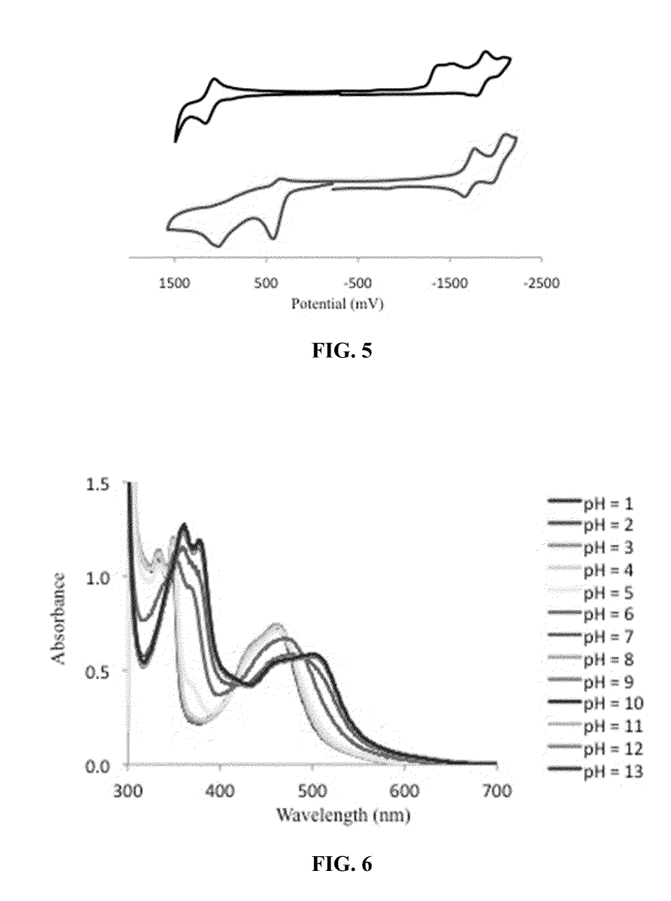

Cyclic voltammetry can elucidate how the ligand interacts with the metal d orbitals in different protonation states, and can therefore explain why the protonated 66′bpy(OH)₂ ligand is more labile in the anti-cancer studies. Cyclic voltammetry data was collected on [Ru(bpy)₂(66′bpy(OH)₂)]²⁺ in acetonitrile solvent with 0.1 M TBAPF₆, FIG. 5. The Ru^{III/II} reduction wave is reversible and occurs at a potential of 1.12 V vs. SCE. This potential is similar to the 1.16 V vs. SCE Ru^{III/II} potential observed for [Ru(bpy)₂(44′bpy(OH)₂)]²⁺ studied previously, indicating the ortho-substituted 66′bpy(OH)₂ ligand can have similar electronic influences on the metal center to the para-hydroxy-substituted 44′bpy(OH)₂ ligand. In addition, both complexes scale with the electron-donation properties of the ligand compared to [Ru(bpy)₃]²⁺ at 1.30 V vs. SCE and [Ru(44′bpy(OH)₂)₃]²⁺ at 0.88 V vs. SCE. By increasing electron-donation to the metal center the Ru^{III} state can be stabilized, thus making the complex easier to oxidize and decreasing the reduction potential. The [Ru(bpy)₂(66′bpy(OH)₂)]²⁺ complex has several reductive and oxidative waves between −1.3 V and −2.1 V vs. SCE associated with ligand redox processes, however, there is significant overlap and individual redox steps cannot be distinguished. There are clear irreversible reductions that have also been observed for [Ru(bpy)₂(44′bpy(OH)₂)]²⁺. These processes contrast with ruthenium complexes containing only methoxy-substituted-bipyridine and unsubstituted-bipyridine ligands which have reversible cyclic voltammograms associated with ligand redox processes.

In order to obtain cyclic voltammetry data of the deprotonated [Ru(bpy)₂(66′bpy(O⁻)₂)] complex, a 5:1 ratio of tetrabutylammonium hydroxide to complex was used. The reductive region of the cyclic voltammogram for [Ru(bpy)₂(66′bpy(O⁻)₂)] becomes reversible upon deprotonation (also observed with the corresponding 4,4′-substituted complex). Two reversible ligand reductions are observed at −1.72 V and −2.04 V vs. SCE and assigned to the unsubstituted bipyridine ligands. These ligand reduction potentials are approximately 0.2 V lower than the potentials observed for deprotonated [Ru(bpy)₂(44′bpy(O⁻)₂)]. Two irreversible oxidative peaks at 0.45 V and 1.03 V vs. SCE were also observed. The tetrabutylammonium hydroxide base is redox active in this region and attempts to subtract the oxidative wave of the base were unsuccessful. As a result it was difficult to determine which of the oxidative waves were associated with the complex in this region. However, the metal-centered Ru^{III/II} reversible wave that appears in the protonated form of the complex is absent as there is no evidence of reversible oxidative processes. This result is indicative of the fact that the metal-centered orbitals mix with the ligand orbitals upon deprotonation, and this was studied by theoretical methods to examine the nature of this mixing. This mixing upon deprotonation, can lead to the more stable metal to ligand bond in the deprotonated from and slows down photo-dissociation compared to the protonated form.

pH-Dependent Changes in Absorbance Spectroscopy

UV/visible absorbance data was collected for [Ru(bpy)₂(66′bpy(OH)₂)]²⁺ in aqueous buffers ranging from pH=1 to pH=13, FIG. 6. The deprotonated form of [Ru(bpy)₂(66′bpy(OH)₂)]²⁺ is red shifted compared to the protonated form of the complex. The pH titration data can show two distinct events that yielded a pKₐ₁=5.26 and pKₐ₂=7.27. This result is in contrast to previous studies carried out with hydroxyl-substituted polypyridyl complexes of ruthenium, whereby the individual deprotonations are not observed and only an average pKₐ value can be reported.

Further analysis of the absorbance spectra in aqueous solution can reveal that the lowest energy MLCT band for the protonated complex is λₘₐₓ=461 nm at pH=1 and shifts approximately 1800 cm⁻¹ to λₘₐₓ=504 nm at pH=13 where the complex can be completely deprotonated. As a comparison, the protonated [Ru(bpy)₂(44′bpy(OH)₂)]²⁺ complex has a λₘₐₓ=462 nm in water and λₘₐₓ=493 nm when deprotonated, a shift of approximately 1300 cm⁻¹. As an additional comparison, another ruthenium complex containing hydroxyl-substituted phenanthroline ligands, [Ru(bpy)₂(47phen(OH)₂)]²⁺ (47phen(OH)₂=4,7-dihydroxy-1,10-phenanthroline) shifts approximately 1500 cm⁻¹ upon deprotonation of the ligand in aqueous solution. All three of the complexes contain two deprotonatable hydroxyl groups.

### Computational Analysis of Electronic Transitions

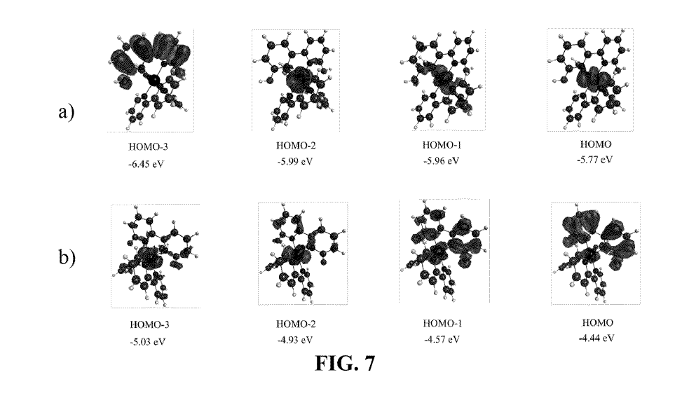

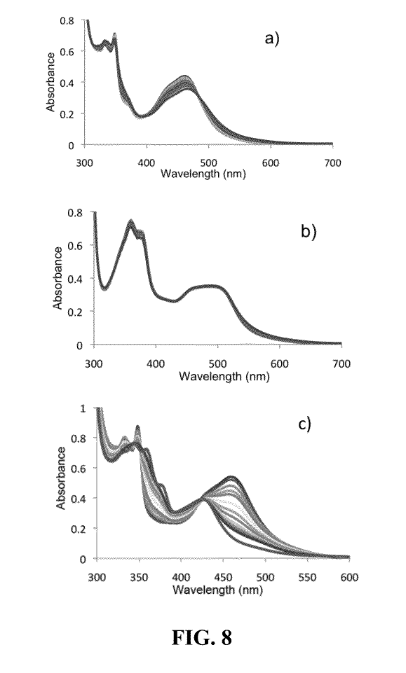

Analysis of the electronic transitions in [Ru(bpy)₂(66′bpy(OH)₂)]²⁺ and [Ru(bpy)₂(66′bpy(O⁻)₂)] was carried out by computational methods using water as the solvent. The four highest occupied molecular orbitals for each complex are depicted in FIG. 7. For the protonated complex, the two lowest energy transitions occur at 411 nm and 405 nm, and are assigned as Metal to Ligand Charge Transfer (MLCT) from a filled metal d orbital to π* orbitals on all three ligands. There is no clear distinction between the bpy and 66′bpy(OH)₂ ligands within these transitions. Following these transitions, there is a gap before two sharp electronic transitions appear between 329 nm and 323 nm. The two sharp electronic transitions are observable in the experimental spectrum of the protonated complex, FIG. 6. These electronic transitions are localized π to π* and Ligand to Ligand Charge Transfer (LLCT) transitions occurring from it molecular orbitals on the 66′bpy(OH)₂ ligand to π* molecular orbitals on both the unsubstituted bpy and 66′bpy(OH)₂ ligands.

Upon deprotonation, computational modeling predicts a red shift as is apparent in the experimental data. The data indicates numerous transitions within the range of 477 nm to 340 nm. These results are apparent in the experimental spectrum, FIG. 6, for [Ru(bpy)₂(66′bpy(O⁻)₂)] which appears as broad over a significant portion of the visible region, with a lowest energy wavelength maxima appearing in the range of 504 nm to 546 nm depending upon the solvent. These transitions are all widely varied originating from pure metal d orbitals, deprotonated ligand orbitals, and orbitals consisting of a mixture metal and deprotonated ligand, FIG. 7. The highest occupied orbitals are higher in energy when compared to the corresponding protonated complex. The Highest Occupied Molecular Orbital (HOMO) and HOMO-1 of the deprotonated complex are mixed metal-deprotonated ligand orbitals, which are in contrast to the protonated complex where no mixing is observed between the metal d orbitals and 66′bpy(OH)₂ ligand. The transitions occur to π* molecular orbitals on the unsubstituted bpy ligand at lower energy and π* molecular orbitals on the deprotonated 66′bpy(O⁻)₂ ligand at higher energy. The electronic transitions to the substituted 66′bpy(O⁻)₂ ligand occur at higher energy due to the more electron-rich nature of the ligand when compared to bpy. The several transitions consist of MLCT, LLCT, and mixed Metal-Ligand to Ligand Charge Transfer (MLLCT). The implications of the significant metal-66′bpy(O⁻)₂ ligand molecular orbital mixing upon deprotonation cannot be understated. From simple bond order analysis, there is a slight increase in the bond order between the metal and 66′bpy(O⁻)₂ ligand that occurs upon deprotonation of the complex (0.4 to 0.5). These new molecular orbitals shared between the metal and ligand result in stronger bonding character between the metal center and the ligand that is not observed in the protonated state where molecular orbital mixing is absent. This result can explain why the complex would photo-dissociate the ligand to a larger extent in the protonated state compared to the deprotonated state.

### Kinetics of Photo-Dissociation Reactions

The rate of ligand loss from [Ru(bpy)₂(66′bpy(OH)₂)]²⁺ to give the active anti-cancer agent ([Ru(bpy)₂(solvent)₂]²⁺) can depend greatly on pH and the solvent composition. At pH 5 in aqueous solution, [Ru(bpy)₂(66′bpy(OH)₂)]²⁺ undergoes blue light induced ligand loss which was monitored by UV/visible spectroscopy over one hour (graph (a) in FIG. 8). At this pH, which is below pKₐ₁ and pKₐ₂, the dihydroxybipyridine ligand should be in its native (protonated) state and should correspond to weaker Ru—N bonds (cf. deprotonated ligand), due to a less electron rich ligand as corroborated by crystallographic and computational results described herein. Analysis of λₘₐₓ=461 nm showed that the decrease in absorbance appears consistent with a zero order process (k_{obs}=1.7(2)×10⁻⁸ M/s, R²=0.986) with an initial rate (0-300 s) that is independent of ruthenium starting material concentration. This A→B process is consistent with the clean isosbestic point observed, and furthermore, a first or second order process can be ruled out due to non-linear (curved) plots for these rate laws (see supplementary materials). Note, that this reaction does appear to slow down slightly from 300-3600 s, and this most likely suggests that an equilibrium is being established due to appreciable back reaction to reform [Ru(bpy)₂(66′bpy(OH)₂)]²⁺.

In contrast, at pH 7.5, there is no measurable photodecomposition of [Ru(bpy)₂(66′bpy(OH)₂)]²⁺ to give the active anti-cancer agent (graph (b) in FIG. 8). At this pH, which is above pKₐ₁ and pKₐ₂, the 6,6′-dihydroxybipyridine ligand should be fully deprotonated, which corresponds to stronger Ru—N bonds. Slight fluctuations in the absorbance values can be attributed to noise, and at this pH the rate of photodecomposition appears to be zero. Thus, the deprotonated, more strongly donating ligand appears to block photodecomposition. Thus, this establishes the framework for how a metal-based prodrug can be selectively activated in cancer cells, which have a lower pH as compared to normal cells.

Studies in acetonitrile were done to monitor the process that gave rise to ([Ru(bpy)₂(CH₃CN)₂]²⁺) and investigate the influence of solvent on the rates. UV/visible studies showed that [Ru(bpy)₂(66′bpy(OH)₂)]²⁺ is stable in acetonitrile solution when protected from light or when exposed to 720 nm red light. In contrast, upon irradiation with blue light (450 nm), [Ru(bpy)₂(66′bpy(OH)₂)]²⁺ in acetonitrile undergoes decomposition to give [Ru(bpy)₂(CH₃CN)₂)]²⁺ and the free ligand, 66′bpy(OH)₂ (λₘₐₓ=343 nm, 421 nm). These products have been prepared independently and their spectra are consistent with those shown for the final result of photodecomposition (graph (c) in FIG. 8). The observed zero order rate constant, k_{obs}=2.6×10⁻⁷ M/s, was determined by fitting an A→B process at 457 nm. The large changes in light absorption show a clean isosbestic point at λ=422 nm and is consistent with an A→B process. Furthermore, the light induced loss of a ligand is consistent with a zero order process that is independent of concentration. The observed rate constant, k_{obs}=2.6(1)×10⁻⁷ M/s, reflects that photodecomposition is nearly complete (87%) in 5 minutes, and it is entirely complete within 20 minutes, graph (c) in FIG. 8. Thus, the reaction proceeds 15 times more quickly in acetonitrile vs. pH 5 aqueous media. This could be attributed to decreased back reaction in acetonitrile, or enhanced stability of the product ligand in acetonitrile since it is better solvated. The product complex is analogous to the likely product of photodecomposition in vivo, namely [Ru(bpy)₂(H₂O)₂)]²⁺.

Labile ligands, like acetonitrile or water, make the products of [Ru(bpy)₂(66′bpy(OH)₂)₂)]²⁺ photodecomposition potentially useful for DNA binding. In a similar fashion, Glazer proposed that [Ru(bpy)₂(66′bpy(Me)₂)]²⁺ loses its bulky 6,6′-dimethyl-bipyridine ligand to form the active drug that binds DNA. (Howerton et al. J. Am. Chem. Soc. 134 (2012) 8324-8327). Studies carried out with [Ru(bpy)₂(44′bpy(OH)₂)]²⁺ reveal no photo-dissociation of ligand under conditions of blue light. Furthermore, the above pH dependent UV-Vis kinetics of photo-dissociation for [Ru(bpy)₂(66′bpy(OH)₂)₂)]²⁺ in blue light clearly show that at pH 7.5 in water the rate of ligand loss is measured as zero, whereas at pH 5 the ligand readily dissociates.

### Summary

The design of prodrugs that can be activated selectively are useful in treating diseases such as cancer. The ability to discriminate between healthy cells and cancer cells could limit some of the adverse effects patients normally suffer from when being treated. Selective targeting of cancer cells can theoretically be achieved with this prodrug approach because cancer cells are typically more acidic than normal cells. Thus, an entirely new approach to selectivity has been demonstrated: a tumor activated prodrug wherein ligand protonation is the activation event. The photo-dissociating ligand can be bound more weakly to the metal center when protonated, and this can allow the active cell-killing agent to be made in greater quantities in cancerous cells. This strategy can be the foundation for the development of a new class of pH sensitive, tumor activated metallo-prodrugs in which, can vary both the pH and the wavelength at which anti-cancer activity is induced.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A metal complex, comprising: at least one metal atom and at least one ligand, wherein the ligand disassociates from the metal atom when the metal complex is contacted with light and is at a pH below 7.

2. The metal complex of claim 1, wherein the ligand is a substituted bipyridine with the following structure: 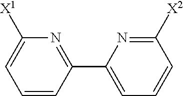
where X¹ and X² are independently selected from —OH, —SH, —NH₂, —SO₃H, —CO₂H, and —SO₂NH₂.

3. The metal complex of claim 1, wherein the ligand is 6,6′-dihydroxy-2,2′-bipyridine.

4. The metal complex of claim 1, wherein the metal atom is selected from the group consisting of ruthenium, platinum, gold, lanthanum, rhodium, silver, iron, zinc, silver, iridium, palladium, osmium, manganese, magnesium, copper, cobalt, scandium, titanium, vanadium, chromium, copper, nickel, yttrium, zirconium, molybdenum, technetium, cadmium, rhenium, and tungsten.

5. The metal complex of claim 1, wherein the metal atom is ruthenium.

6. The metal complex of claim 1, further comprising a counterion.

7. The metal complex of claim 6, wherein the counterion is selected from the group consisting of chloride, bromide, iodide, hexaflurophoshate, tetrafluroborate, carbonate, hydroxide, lithium, and sodium.

8. The metal complex of claim 1, further comprising a ligand selected from the group consisting of phenanthroline, biphenyl, anthracene, naphthalene, dipyrido[3,2-f:2′,3′-h]-quinoxaline), dipyrido[3,2-a:2′,3′-c]phenazine, and 3,6-bis(2′-pyridyl)pyridazine.

9. The metal complex of claim 1, wherein the metal complex is selected from the group consisting of [Ru(bpy)₂(66′bpy(OH)₂)][PF₆]₂.H₂O, [Ru(bpy)₂(66′bpy(OH)₂)][PF₆]₂, and [Ru(bpy)₂(66′bpy(O⁻)₂)].

10. An anti-cancer drug, comprising: a metal complex according to claim 1.

11. The anti-cancer drug of claim 10, wherein the ligand intercalates a nucleic acid molecule.

12. The anti-cancer drug of claim 10, wherein the metal complex is selected from the group consisting of [Ru(bpy)₂(66′bpy(OH)₂)][PF₆]₂.H₂O, [Ru(bpy)₂(66′bpy(OH)₂)][PF₆]₂, and [Ru(bpy)₂(66′bpy(O⁻)₂)].

13. A metal complex having Formula I: 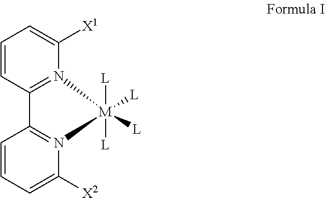
where M is a metal, X¹ and X² are protic groups, and each L is either a monodentate ligand or together with another L a bidentate ligand.

14. The metal complex of claim 13, wherein M is ruthenium or platinum.

15. The metal complex of claim 13, wherein X¹ and X² are independently, —OH, —SH, —NH₂, —SO₃H, —CO₂H, or —SO₂NH₂.

16. The metal complex of claim 15, wherein X¹ and X² are OH.

17. The metal complex of claim 13, wherein two L groups are substituted or unsubstituted bipyridinyl or two L groups are unsubstituted or unsubstituted phenanthroline, where when substituted they are substituted with alkyl, alkoxy, alkenyl, alkynyl, aryl, heteroaryl, aldehyde, amino, carboxylic acid, ester, ether, halide, hydroxy, ketone, nitro, silyl, sulfo-oxo, or thiol.

18. A method of killing a cancer cell in a subject comprising: administering to the subject a compound of claim 1 and then irradiating the cancer cell with light.

19. The method of claim 18, wherein the cancer is breast cancer, ovarian cancer, bladder cancer, cervical cancer, gastrointestinal cancer, genitourinary cancer, head and neck cancer, lung cancer, pancreatic cancer, prostate cancer, renal cancer, skin cancer, or testicular cancer.
